(19) United States
(12) Patent Application Publication
(10) Pub. No.: US 2016/0073998 A1
(43) Pub. Date: Mar. 17, 2016

(54) X-RAY DIAGNOSTIC APPARATUS

(71) Applicants: KABUSHIKI KAISHA TOSHIBA, Minato-ku (JP); Toshiba Medical Systems Corporation, Otawara-shi (JP)

(72) Inventors: Kazunari SHIMA, Nasushiobara (JP); Masanori MATSUMOTO, Nasushiobara (JP); Yoshimasa KOBAYASHI, Nasushiobara (JP); Yusuke NARABU, Nasushiobara (JP)

(73) Assignees: KABUSHIKI KAISHA TOSHIBA, Minato-ku (JP); Toshiba Medical Systems Corporation, Otawara-shi (JP)

(21) Appl. No.: 14/846,079

(22) Filed: Sep. 4, 2015

(30) Foreign Application Priority Data
Sep. 17, 2014 (JP) 2014-189328

(51) Int. Cl. A61B 6/00

(52) U.S. Cl. CPC A61B 6/547; A61B 6/487; A61B 6/542; A61B 6/548

(57) ABSTRACT

According to one embodiment, an X-ray diagnostic apparatus includes an radiography system radiographs the subject, a support frame movably supports the radiography system, a communication circuitry obtains skin dose information of the subject, a processing circuitry decides the irradiation range at the movement destination of the radiography system based on the skin doses of the subject, a control circuitry controls the support frame to move the radiography system to an radiography position corresponding to the decided irradiation range at the movement destination. The irradiation range at the movement destination comes close to the irradiation range on the subject immediately before the movement, and a skin dose corresponding to the irradiation range at the movement destination is less than a threshold.

### CROSS-REFERENCE TO RELATED APPLICATIONS

This application is based upon and claims the benefit of priority from the prior Japanese Patent Application No. 2014-189328, filed Sep. 17, 2014 the entire contents of which are incorporated herein by reference.

### FIELD

Embodiments described herein relate generally to an X-ray diagnostic apparatus.

### BACKGROUND

Catheter treatment imposes a lighter burden on patients than surgical treatment, and hence is one of effective medical treatments. In catheter treatment using an X-ray diagnostic apparatus, X-ray fluoroscopy is often performed. A user such as a doctor performs catheter treatment while seeing a fluoroscopic image concerning a subject which is obtained by X-ray fluoroscopy and updated in real time. X-ray fluoroscopy is a radiography method of continuously or intermittently irradiating a subject with a lower dose of X-rays than X-ray radiography in one-shot imaging. However, in catheter treatment requiring a long treatment time, even X-ray fluoroscopy which irradiates a subject with a low dose of X-rays has a problem that a skin dose corresponding to an X-ray irradiation range increases with the lapse of time. As one technique for solving such a problem, there is available a technique of rotating an imaging direction through 180° when the skin dose reaches a given threshold. This technique can prevent partial and excessive exposure of a patient to X-rays while suppressing a deterioration in the procedure efficiency of a user such as a doctor because the change between fluoroscopic images obtained before and after the change of the imaging direction is small.

In order to rotate the imaging direction through 180°, however, an X-ray diagnostic apparatus, if it includes an X-ray tube and an X-ray detector so as to make them face each other, needs to rotate the C-arm through 180°. When rotating this C-arm, it is inefficient to move a subject or person who can interfere with the C-arm for only the rotation of the C-arm.

### BRIEF DESCRIPTION OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of an X-ray diagnostic apparatus 1 according to an embodiment;

FIG. 2 is a view showing an example of a screen indicating skin dose information of a subject;

FIG. 3 is the first supplementary explanatory view for explaining a method of deciding an irradiation range at a movement destination by an irradiation range decision unit according to the embodiment;

FIG. 4 is the second supplementary explanatory view for explaining a method of deciding an irradiation range at a movement destination by the irradiation range decision unit according to the embodiment;

FIG. 5 is the third supplementary explanatory view for explaining a method of deciding an irradiation range at a movement destination by the irradiation range decision unit according to the embodiment;

FIG. 6 is a supplementary explanatory view for explaining a method of inputting a tilting direction by the user according to the embodiment;

FIG. 7 is the fourth supplementary explanatory view for explaining a method of deciding an irradiation range at a movement destination by the irradiation range decision unit according to the embodiment;

FIG. 8 is the first supplementary explanatory view for explaining processing by radiographic control circuitry according to the embodiment;

FIG. 9 is the second supplementary explanatory view for explaining processing by the radiographic control circuitry according to the embodiment; and

FIG. 10 is a flowchart for explaining a workflow using the X-ray diagnostic apparatus according to the embodiment.

### DETAILED DESCRIPTION

In general, according to one embodiment, an X-ray diagnostic apparatus includes a radiography system, a support frame, communication circuitry, processing circuitry, and control circuitry. The radiography system includes an X-ray tube which generates X-rays and an X-ray detector which detects the X-rays generated from the X-ray tube and transmitted through a subject, and images the subject. The support frame movably supports the radiography system. The communication circuitry obtains skin dose information of the subject. The processing circuitry decides the irradiation range at the movement destination of the radiography system based on the skin doses of the subject. The control circuitry controls the support frame to move the radiography system to a radiography position corresponding to the decided irradiation range at the movement destination. The irradiation range at the movement destination comes close to the irradiation range on the subject immediately before the movement, and a skin dose corresponding to the irradiation range at the movement destination is less than a threshold.

An X ray diagnostic apparatus according to this embodiment will be described below with reference to the accompanying drawing. Note that the same reference numerals in the following description denote constituent elements having almost the same functions and arrangements, and a repetitive description will be made only when required.

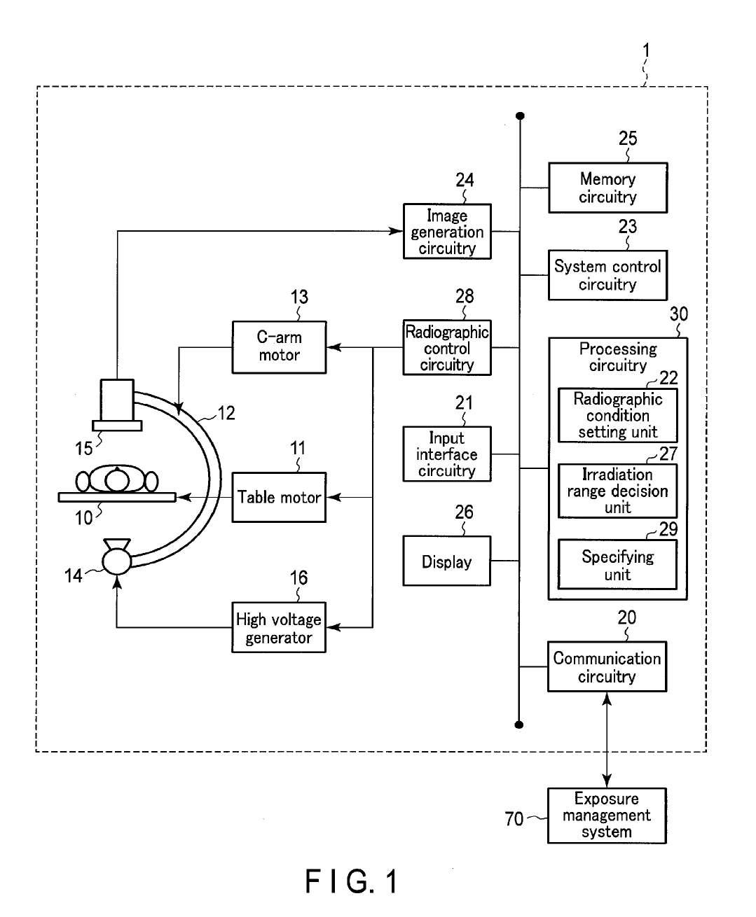

FIG. 1 is a block diagram showing an example of an X-ray diagnostic apparatus according to this embodiment. An X-ray diagnostic apparatus 1 according to the embodiment includes a gantry and a data processing apparatus. The gantry includes a bed 10, a bed motor 11, a C-arm 12, a C-arm motor 13, an X-ray generator 14, an X-ray detector 15, and a high voltage generator 16.

The bed 10 movably supports a top plate. A subject is placed on the top plate. The bed 10 moves the top plate when the bed motor 11 is driven under the control of radiographic control circuitry 28.

The C-arm 12 supports the radiography system. The radiography system includes the X-ray generator 14 and the X-ray detector 15. The radiography system radiographs an ROI (Region Of Interest) of the subject placed on the top plate. The C-arm 12 supports the X-ray generator 14 on its one end and the X-ray detector 15 on the other end. The C-arm 12 supports the X-ray generator 14 and the X-ray detector 15 so as to make them face each other.

The X-ray generator 14 includes an X-ray tube, an X-ray filter, and an X-ray collimator. The X-ray tube is a vacuum tube which generates X-rays. The X-ray tube generates X-rays from the focus upon receiving a high voltage (tube voltage) and a tube current from the high voltage generator 16. Generated X-rays are transmitted through the X-ray filter and formed into a beam by the X-ray collimator. The X-ray filter is arranged to, for example, reduce the X-ray exposure dose of a subject and improve image quality. For example, the X-ray filter removes long-wavelength components unnecessary for diagnosis from the continuous spectrum of X-rays generated from the X-ray focus. The X-ray collimator limits an X-ray irradiation range under the control of the radiographic control circuitry 28. This can prevent an increase in the exposure dose of a patient when the X-rays generated from the X-ray focus are applied outside the imaging range desired by a user such as a doctor.

The X-ray detector 15 includes a plurality of X-ray detection elements. The plurality of X-ray detection elements are arranged in a two-dimensional array. The two-dimensional array detector is called an FPD (Flat Panel Detector). Each element of the FPD detects the X-rays generated from the X-ray generator 14 and transmitted through a subject. Each element of the FPD outputs an electrical signal corresponding to a detected X-ray intensity. Note that the X-ray detector 15 may be formed from a combination (I.I-TV) of an image intensifier and a TV camera instead of the FPD described above. A line connecting the focus of the X-ray tube and the center of the detection surface of the X-ray detector 15 will be referred to as an imaging axis, and a corresponding direction will be referred to as an imaging direction.

The C-arm 12 is movably supported by a C-arm support frame. The C-arm support frame has a plurality of rotation shafts for the movement of the C-arm 12. The respective mechanisms constituting the C-arm support frame are rotated when the C-arm motor 13 is driven under the control of the radiographic control circuitry 28. The rotating operations of the respective mechanisms constituting the C-arm support frame can rotate the C-arm 12 about the plurality of rotation shafts. Rotating the C-arm 12 about the plurality of rotation shafts makes it possible to freely change the imaging direction with respect to a region of interest of a subject.

Note that this embodiment has exemplified the C-arm 12 as a mechanism which supports the X-ray generator 14 and the X-ray detector 15. However, this mechanism is not limited to the C-arm 12 as long as the mechanism can support the X-ray generator 14 and the X-ray detector 15 so as to make them face each other and freely move (rotate) the imaging axis with respect to a subject. For example, the C-arm 12 and the C-arm support frame can be replaced with a ceiling-mounted Ω-arm and an Ω-arm support frame. In addition, the C-arm 12 and the C-arm support frame can be replaced with the first support frame which movably supports the X-ray generator 14 and the second support frame which movably supports the X-ray detector 15. In this case, for example, the first support frame has a mechanism which can be mounted on the floor. The second support frame has a mechanism which can be suspended from the ceiling. In this case, the first support frame may include a mechanism capable of translating with respect to the floor surface and a mechanism capable of moving vertically with respect to the floor surface. In addition, the second support frame may include a mechanism capable of translating with respect to the ceiling surface and a mechanism capable of moving vertically with respect to the ceiling surface. The first and second support frames support the X-ray generator 14 and the X-ray detector 15 so as to make them face each other. It is possible to change the imaging axis with respect to a subject by synchronously controlling the moving operations of the first and second support frames.

The data processing apparatus includes communication circuitry 20, input interface circuitry 21, system control circuitry 23, image generation circuitry 24, memory circuitry 25, a display 26, processing circuitry 30, and radiographic control circuitry 28.

The communication circuitry 20 (also called transmission/reception circuitry or acquisition circuitry) is a communication interface with an exposure management system 70 connected to the X-ray diagnostic apparatus 1. The communication circuitry 20 transmits, to the exposure management system 70, the fluoroscopy conditions set by the system control circuitry 23 and the geometric positional relationship between the C-arm 12 and the top plate. The exposure management system 70 generates data concerning the skin doses of the subject based on the fluoroscopy conditions and the geometric positional relationship between the C-arm 12 and the top plate. Therefore, the data concerning the skin doses of the subject is an estimated value (reference value) of an incident skin dose corresponding to a range in which the subject is irradiated with X-rays. The communication circuitry 20 receives data concerning the skin doses of the subject from the exposure management system 70. In addition, the communication circuitry 20 may receive, from the exposure management system 70, display screen data three-dimensionally representing a skin dose color map by reflecting estimated values of the incident skin doses of the subject in a patient model. In addition, the communication circuitry 20 outputs a signal notifying the start and end of X-ray fluoroscopy to the exposure management system 70.

The input interface circuitry 21 is a user interface with which the user inputs instruction information to the X-ray diagnostic apparatus 1. More specifically, instruction information includes fluoroscopy conditions and movement conditions. In this cased, the fluoroscopy conditions include a tube voltage, tube current, irradiation time, SID (Source to Image-receptor Distance: the distance between the X-ray detection surface and the X-ray focus), ROI, and FOV (Field Of View). Note that the fluoroscopy conditions may include information concerning a region subjected to fluoroscopy. More specifically, the initial values of a tube voltage, tube current, and irradiation time are automatically set in accordance with patient information, the type of procedure, and the like. X-ray radiography is then performed to adjust the above conditions before the start of the procedure. A user such as a doctor sets a tube voltage, tube current, irradiation time, SID, ROI, FOV, and the like while checking a fluoroscopic image. Therefore, for example, the user moves the C-arm 12, the top plate, and the like so as to make the central position of the ROI overlap the central position of the fluoroscopic image while checking the fluoroscopic image. In this case, the positions of the respective mechanisms are preferably set to make the central position of the ROI overlap the isocenter. The user then adjusts the SID while checking the ROI and sets an FOV. Note that an ROI is set in accordance with user operations on a fluoroscopic image corresponding to the front side of the subject and a fluoroscopic image corresponding to a side surface side of the subject. Movement conditions are conditions concerning a method of deciding an irradiation range at a movement destination by an imaging direction decision unit (to be described later). The movement conditions include the moving velocity of the C-arm 12, a threshold for skin doses (to be referred to as a dose threshold hereinafter), and a method of deciding an irradiation range at a movement destination. The input interface circuitry 21 includes, for example, input devices such as a mouse, keyboard, trackball, touch panel, and switches. For example, the user can input instruction information to the X-ray diagnostic apparatus by operating these input devices. In addition, the input interface circuitry 21 includes a fluoroscopy switch. The fluoroscopy switch is, for example, a foot switch. While the fluoroscopy switch is stepped on, the X-ray fluoroscopy mode is kept on.

The system control circuitry 23 includes, as hardware resources, a processing device (processor) such as a CPU (Central Processing Unit) or MPU (Micro Processing Unit), and storage devices (memories) such as a ROM and RAM. In addition, the system control circuitry 23 may be implemented by an ASIC, FPGA, CPLD, or SPLD. The system control circuitry 23 temporarily stores the information input to the X-ray diagnostic apparatus 1 in a semiconductor memory via the input interface circuitry 21. The system control circuitry 23 comprehensively controls the respective units of the X-ray diagnostic apparatus 1 based on the input information.

The image generation circuitry 24 executes preprocessing for an output signal from the X-ray detector 15. Preprocessing includes various types of correction processing, amplification processing, and A/D conversion processing. The image generation circuitry 24 generates X-ray image data based on data after preprocessing which corresponds to the output signal from the X-ray detector 15. The X-ray image obtained under X-ray fluoroscopy, in particular, will be referred to as a fluoroscopic image. A pixel value assigned to each pixel of an X-ray image is, for example, a value corresponding to an X-ray attenuation coefficient concerning a substance on the transmission path of X-rays.

The memory circuitry 25 includes a semiconductor memory device such as a flash SSD (Solid State Drive) as a semiconductor memory element and an HDD (Hard Disk Drive). The memory circuitry 25 stores the X-ray image (fluoroscopic image) data generated by the image generation circuitry 24.

The display 26 displays a fluoroscopic image. The display 26 also displays the skin dose information of a subject which are obtained via the communication circuitry 20. At this time, the display 26 may display the display screen of the exposure management system 70.

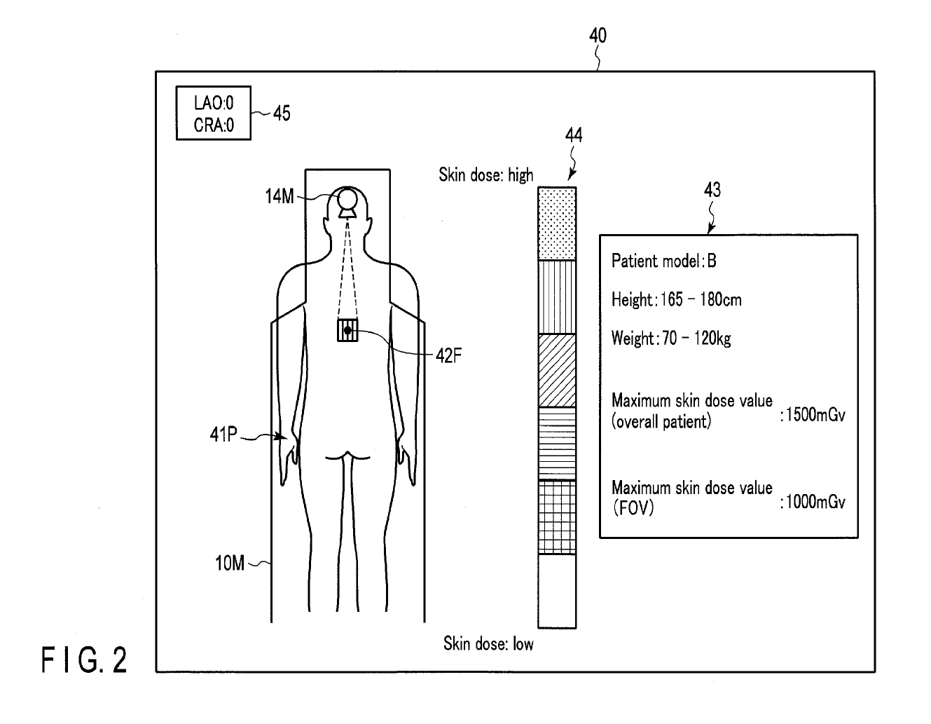

FIG. 2 is a view showing an example of the screen indicating information concerning the skin doses of a subject. FIG. 2 shows how a patient is irradiated with X-rays from the rear surface of the top plate. A top plate model 10M, an X-ray tube model 14M, a patient model 41P, a patient information display box 43, a dose bar graph 44, and an imaging direction 45 are displayed on a display screen 40 of the display 26. They can be arranged in accordance with the layout of the exposure management system 70 or may be arranged in accordance with the layout set by the X-ray diagnostic apparatus based on data. The patient information display box 43 includes a plurality of items concerning a patient. As shown in FIG. 2, the plurality of items include a patient model, height, weight, the maximum skin dose value of the overall patient, and the maximum skin dose value in an FOV. The patient model indicates the type of the patient model 41P. In this case, the patient model 41P is of type B. The height and weight indicate ranges corresponding to type B of the patient model 41P. A color map and the patient model 41P are displayed while the color map corresponding to skin dose values is superimposed the patient model 41P. The dose bar graph 44 indicates the correspondence relationship between the skin doses and colors (graphic patterns). The current irradiation range is indicated as an irradiation range 42F. A user such as a doctor can intuitively check the exposure state of the patient by seeing the patient model 41P on which the color map corresponding to the skin dose values is superimposed.

Note that the display screen 40 may display an irradiation range at the movement destination which indicates a range in which the radiography system is moved.

The processing circuitry 30 includes, as hardware resources, a processing device (processor) such as a CPU or MPU, and storage devices (memories) such as a ROM and RAM. In addition, the processing circuitry 30 may be implemented by an ASIC, FPGA, CPLD, or SPLD. The processing device implements the functions of an radiographic condition setting unit 22, an irradiation range decision unit 27, and a specifying unit 29 by reading out and executing programs stored in the storage device. Note that programs may be directly incorporated in the circuitry of the processing device instead of being stored in the storage device. In this case, the processing device implements the functions of the radiographic condition setting unit 22, the specifying unit 29, and the irradiation range decision unit 27 by reading out and executing programs incorporated in the circuitry. Alternatively, the processing circuitry 30 may incorporate dedicated hardware circuitry functioning as the radiographic condition setting unit 22, dedicated hardware circuitry functioning as the irradiation range decision unit 27, and dedicated hardware circuitry functioning as the specifying unit 29.

The radiographic condition setting unit 22 sets the fluoroscopy conditions and movement conditions input via the input interface circuitry 21. The specifying unit 29 specifies a range in which the skin doses of the subject are less than a preset dose threshold from the estimated values of the incident skin doses of the subject which are obtained via the communication circuitry 20.

The irradiation range decision unit 27 decides an irradiation range at a movement destination based on the skin doses of the subject input via the communication circuitry 20. The irradiation range decision unit 27 decides an irradiation range at the movement destination to change the irradiation range during X-ray fluoroscopy. The imaging control circuitry 28 (to be described later) moves the radiography system in response to a timing when a skin dose in the current irradiation range has reached the dose threshold. More specifically, the imaging control circuitry 28 moves the radiography system in response to a timing when even part of the current irradiation range has reached the dose threshold. Note that the current irradiation range is the irradiation range in which the radiography system performs imaging immediately before the movement of the radiography system to the irradiation range at the movement destination. The current irradiation range will be referred to as the immediately preceding irradiation range hereinafter. Therefore, a skin dose in the immediately preceding irradiation range at the timing of the movement of the radiography system is a dose threshold.

The irradiation range decision unit 27 decides an irradiation range at a movement destination so as to satisfy at least the following two requirements.

(1) A skin dose value in an irradiation range at a movement destination is smaller than a dose threshold.

As the dose threshold in this case, the maximum allowable skin dose value of a subject is preferably used. However, a dose threshold can be changed as needed in accordance with a user instruction.

(2) An irradiation range at a movement destination is close to an immediately preceding irradiation range.

In this case, the phrase “is close” indicates that the distance between the irradiation range at the movement destination and the immediately preceding irradiation range is short. In other words, the imaging direction corresponding the irradiation range at the movement destination (to be referred to as the imaging direction at the movement destination) is close to the imaging direction corresponding to the immediately preceding irradiation range (to be referred to as the immediately preceding imaging direction hereinafter). The phrase “is close” in this case indicates that the angle defined by the imaging direction at the movement destination and the immediately preceding imaging direction is small.

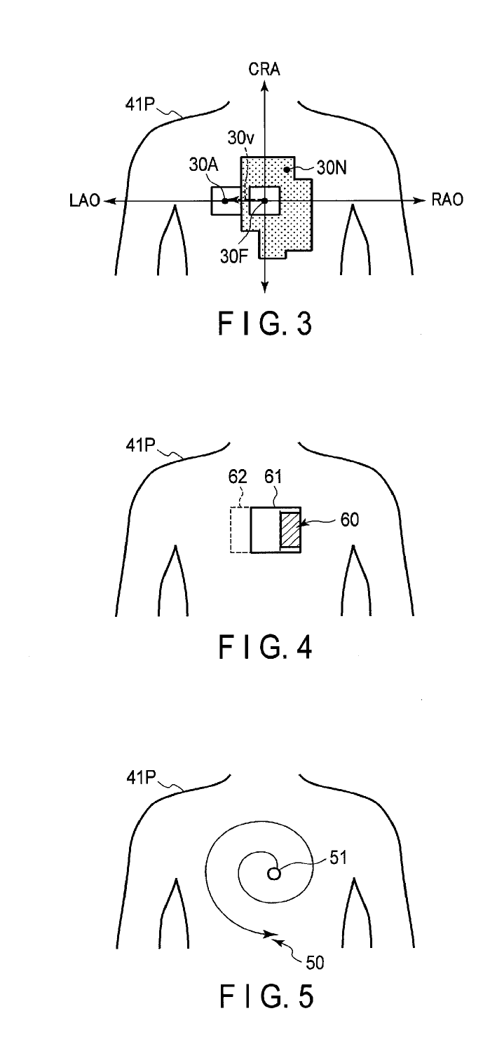

A method of deciding an irradiation range at a movement destination by the irradiation range decision unit 27 will be described next with reference to FIGS. 3, 4, 5, 6, and 7.

FIG. 3 is the first supplementary explanatory view for explaining the method of deciding an irradiation range at a movement destination by the irradiation range decision unit 27 according to the embodiment. FIGS. 3, 4, 5, and 7 described below each schematically show the back side (top plate rear surface) of the subject placed on his/her back on the top plate, showing part of the patient model 41P described with reference to FIG. 2. A color map corresponding to skin dose values is superimposed on the patient model 41P. On the color map shown in FIG. 3, a range in which skin doses are equal to or more than the dose threshold is represented as a partial range 30N. A current (immediately preceding) irradiation range on the patient model 41P is represented as an irradiation range 30F. The irradiation range decision unit 27 decides an irradiation range at a movement destination so as to satisfy at least the requirements (1) and (2).

First of all, the specifying unit 29 specifies a range (to be referred to as a low dose range hereinafter) whose skin doses are lower than the dose threshold based on the skin doses of the subject input via the communication circuitry 20. Referring to FIG. 3, since the range whose skin doses are equal to or more than the dose threshold is the partial range 30N, the specifying unit 29 specifies a range, of the entire range on the skin surface of the subject, which is other than the partial range 30N as a low dose range.

The irradiation range decision unit 27 then decides an irradiation range at the movement destination from the low dose range specified by the specifying unit 29. Imaging the same ROI in different imaging directions leads to changes in the shape, position, and size of the irradiation range. The irradiation range decision unit 27 specifies the shape, position, and size of an irradiation range at the movement destination based on the mechanical positional relationship between the radiography system at the movement destination and the top plate and the like. The irradiation range decision unit 27 then decides, as an irradiation range at the movement destination, a range which is included in the low dose range and is closest to the immediately preceding irradiation range 30F. In this manner, the irradiation range decision unit 27 decides an irradiation range 30A at the movement destination which is located at a shortest distance 30v from the immediately preceding irradiation range 30F. Note that since the skin dose of the entire immediately preceding irradiation range is the dose threshold, the immediately preceding irradiation range 30F is not included in the low dose range. Therefore, the irradiation range at the movement destination does not overlap the immediately preceding irradiation range.

FIG. 4 is the second supplementary explanatory view for explaining a method of deciding an irradiation range at a movement destination by the irradiation range decision unit 27 according to the embodiment. On the color map shown in FIG. 4, a range which is less than a dose threshold and has a predetermined skin dose value is represented as a partial range 60. On the patient model 41P, an immediately preceding irradiation range is represented as an irradiation range 61. The irradiation range decision unit 27 decides an irradiation range at a movement destination so as to satisfy at least the requirements (1) and (2).

First of all, the specifying unit 29 specifies a low dose range based on the skin doses of the subject input via the communication circuitry 20. A skin dose corresponding to the partial range 60 in the immediately preceding irradiation range 61 reaches the dose threshold earlier than a range other than the immediately preceding irradiation range. Therefore, the specifying unit 29 specifies, as a low dose range, a range, of the entire range on the skin surface of the subject, which is other than the partial range 60.

The irradiation range decision unit 27 then decides an irradiation range at the movement destination from the low dose range specified by the specifying unit 29. Imaging the same ROI in different imaging directions leads to changes in the shape, position, and size of the irradiation range. The irradiation range decision unit 27 specifies the shape, position, and size of an irradiation range at the movement destination based on the mechanical positional relationship between the radiography system at the movement destination and the top plate and the like. The irradiation range decision unit 27 then decides, as an irradiation range at the movement destination, a range which is included in the low dose range and is closest to the immediately preceding irradiation range 61. In this manner, the irradiation range decision unit 27 decides an irradiation range 62 at the movement destination. As described above, if the immediately preceding irradiation range 61 has a partially different dose distribution, the irradiation range at the movement destination may partially overlap the immediately preceding irradiation range.

Note that the irradiation range decision unit 27 may decide an irradiation range at the movement destination so as to satisfy a requirement (3) described below in addition to the requirements (1) and (2) described above.

(3) As the direction from an immediately preceding irradiation range to an irradiation range at a movement destination, the direction (to be referred to as the tilting direction hereinafter) set with reference to the immediately preceding irradiation range is decided.

A method of deciding a tilting direction will be described below.

A tilting direction is decided in accordance with a user instruction before the start of a procedure. In this case, the user can decide a direction to tilt the radiography system in accordance with a procedure environment at the start of the procedure. Note that a tilting direction may be decided in accordance with a predetermined changing route of an irradiation range. In this case, the memory circuitry 25 stores data concerning the changing route of the irradiation range.

FIG. 5 is the third supplementary explanatory view for explaining a method of deciding an irradiation range at a movement destination by the irradiation range decision unit 27 according to this embodiment. The irradiation range decision unit 27 decides a tiling direction based on the preset changing route of the irradiation range. More specifically, the irradiation range decision unit 27 decides an irradiation range at a movement destination based on the immediately preceding irradiation range, the history of change of the irradiation range, and the changing route of the irradiation range. FIG. 5 shows an example of the changing route of an irradiation range. As shown in FIG. 5, a changing route 50 of an irradiation range has a spiral form. A maker 51 corresponds to a reference point of an irradiation range. The irradiation range decision unit 27 records the initially set central position of an irradiation range as a reference point. The irradiation range decision unit 27 then decides a tilting direction, with the reference point being a start point, in accordance with the shape of the changing route 50. The central position of the irradiation range decided in this manner is set to increase the distance from the reference point. In this case, the irradiation range decision unit 27 decides an irradiation range at the movement destination so as to satisfy the requirements (1), (2), and (3). That is, the irradiation range decision unit 27 decides an irradiation range at the movement destination so as to avoid the irradiation range at the movement destination from including a range having a skin dose equal to or more than a dose threshold.

This allows the user to know the moving direction of the radiography system in advance and move any devices that can interfere with the radiography system before the start of a procedure. It is therefore possible to obtain the effect of reducing the risk of interference between the radiography system and others. In addition, when the predetermined changing route of the irradiation range has a spiral form or the like with the distance from the reference point gradually increasing as shown in FIG. 5, the angle change between fluoroscopic images before and after the movement of the C-arm 12 is small, and the angle change from the fluoroscopic image at initial settings can be suppressed. This allows the user to see a fluoroscopic image with a small change from the fluoroscopic image at initial settings. Therefore, the user can maintain the procedure efficiency.

A tilting direction may be decided in accordance with a user instruction during X-ray fluoroscopy. This allows the user to decide the tilting direction of the radiography system in accordance with a procedure environment during X-ray fluoroscopy. More specifically, a user such as a doctor can decide the tiling direction of the radiography system in consideration of the standing position of the user at the time of the movement of the radiography system, the placement of devices necessary for a procedure, and the like. This can reduce the risk that the radiography system will interfere with other persons (things). An imaging direction that makes it easy to perform a procedure depends on a region subjected to the procedure and the orientation of a device such as an inserted catheter. For example, when operating the catheter, it is preferable to set an imaging direction at a position near a direction perpendicular to the inserting direction of the catheter. This is because a fluoroscopic image corresponding to the direction perpendicular to the inserting direction of the catheter is an image properly reflecting the shade of the catheter. This can improve the operability of the catheter by the user. Therefore, the user can decide the tilting direction of the radiography system in consideration of the state of a procedure or procedure environment at the time of the movement of the radiography system, thereby obtaining the effect of maintaining the procedure efficiency.

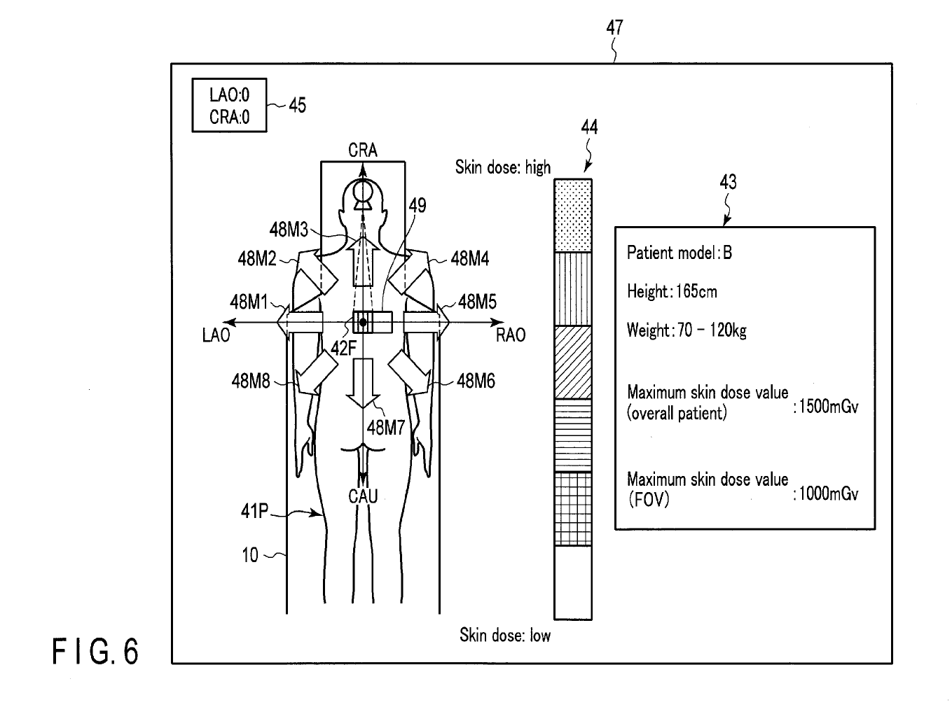

FIG. 6 is a supplementary explanatory view for explaining a method of inputting a tilting direction by the user according to this embodiment. FIG. 6 shows an input screen for inputting the tilting direction displayed on the display 26. As shown in FIG. 6, the display screen 40 of the exposure management system 70 described with reference to FIG. 2 displays a plurality of marks (marks 48M1 to 48M8) for inputting a tilting direction around the current irradiation range 42F. In addition, the display screen 40 displays an irradiation range 49 at a movement destination. The user can input a tilting direction by selecting a mark, of the plurality of displayed marks, which corresponds to the direction in which he/she wants to tilt the imaging direction. If, for example, the mark 48M5 is selected, since the tilting direction coincides with the RAO direction, the direction tilted from the current imaging direction to the RAO direction is decided as the imaging direction at the movement destination.

Note that the user may input a tilting direction on the screen displayed on the display of the exposure management system 70. In this case, an input screen identical or similar to an input screen 47 is displayed on the screen of the exposure management system 70. The user inputs a tiling direction on the screen of the exposure management system 70. Data concerning the tilting direction is input to the X-ray diagnostic apparatus 1 via the communication circuitry 20.

A tilting direction may be decided such that the imaging direction at the movement destination is close to the imaging direction (to be referred to as the initial imaging direction hereinafter) corresponding to the initially set irradiation range. In general, the initial imaging direction is set in a direction in which the user wants most to observe an ROI. Therefore, it is possible to obtain the effect of maintaining the easiness of observation of the ROI by the user by deciding a direction close to the initially set imaging direction as an imaging direction at the movement destination.

A tilting direction may be decided such that the imaging direction at the movement destination is close to the zenith direction (a direction perpendicular to the top plate). In general, when the imaging direction is parallel to the zenith direction, the risk of contact between the radiography system and the top plate or the like is low. That is, this placement can be said to be safe. Therefore, deciding a direction close to the zenith direction as an imaging direction at the movement destination can reduce the risk that the radiography system will interfere with others.

As a tilting direction, a direction in which the area of a low dose range is large may be decided with reference to the immediately preceding irradiation range. This will be described in the case of, for example, the skin dose distribution shown in FIG. 3. In this case, the range of the subject P is divided in the RAO, LAO, CRA, and CAU directions with reference to the immediately preceding irradiation range 30F. For example, a low dose range corresponding to the RAO direction is a range, of the two ranges divided from the partial range 30N along the CRA/CAU axis, which corresponds to the RAO direction. The irradiation range decision unit 27 calculates the total areas of low dose ranges corresponding to the respective directions. The irradiation range decision unit 27 then decides, as a tilting direction, a direction in which the low dose range has the largest total area. For example, in the case shown in FIG. 3, the irradiation range decision unit 27 decides, as a tiling direction, the LAO direction in which the low dose range has the largest area among the RAO, LAO, CRA, and CAU directions. This makes it possible to distribute a wide low dose range around the immediately preceding irradiation range. Therefore, when the procedure time is long and the irradiation range must be changed several times, it is highly possible to set an irradiation range at a movement destination at a position near the immediately preceding irradiation range. This can suppress the angle change between fluoroscopic images before and after movement, thereby obtaining the effect of maintaining the procedure efficiency of the user.

Several tilting direction decision methods have been described above. The tilting direction decision method to be used can be changed as needed in accordance with a user instruction via the input interface circuitry 21. In addition, priority levels may be assigned to the respective tilting direction decision methods.

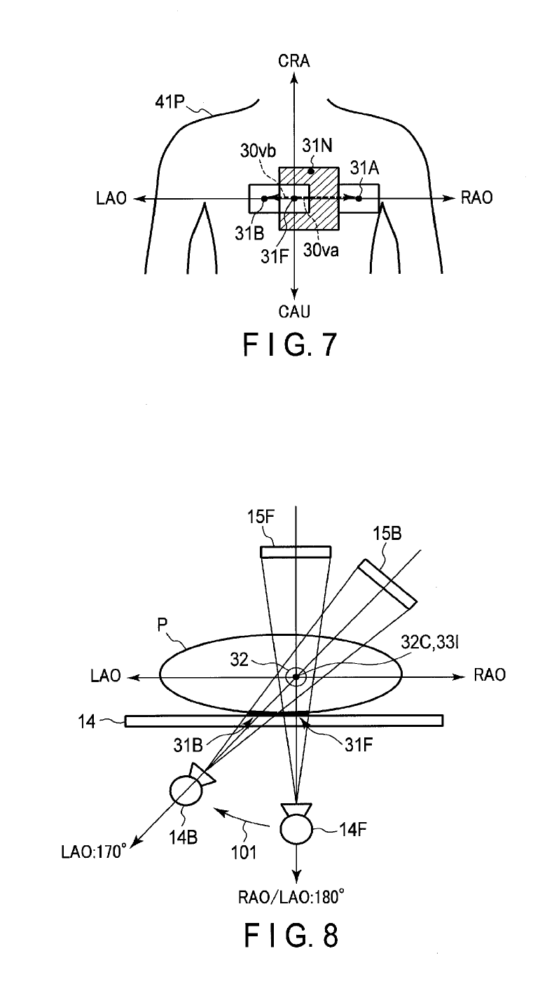

FIG. 7 is the fourth supplementary explanatory view for explaining a method of deciding an irradiation range at a movement destination by the irradiation range decision unit 27 according to this embodiment. On the color map shown in FIG. 7, a range in which skin doses are equal to or more than a dose threshold is represented as a partial range 31N. A current (immediately preceding) irradiation range on the patient model 41P is represented as an irradiation range 31F. The irradiation range decision unit 27 decides an irradiation range at a movement destination so as to satisfy the requirements (1), (2), and (3). In this case, priority levels may be set for the requirements (2) and (3). The priority level settings can be changed as needed in accordance with a user operation via the input interface circuitry 21. That is, the user selects between deciding an irradiation range at a movement destination with priority being given to a tilting direction and deciding an irradiation range at a movement destination with priority being given to the distance from the immediately preceding irradiation range.

Assume a case in which priority is given to the requirement (2). In this case, the irradiation range decision unit 27 decides an irradiation range 31B as an irradiation range at the movement destination. The irradiation range 31B at the movement destination is an irradiation range from a shortest distance 30vb from the immediately preceding irradiation range 31F. Note that when priority is given to the requirement (2), if there are a plurality of irradiation ranges at the movement destination, the requirement (3) is applied. In this case, the irradiation range decision unit 27 decides, as an irradiation range at the movement destination, an irradiation range, of a plurality of irradiation range candidates at the movement destination, which is closest to the set tiling direction.

Assume a case in which priority is given to the requirement (3). Assume that in this case, as described with reference to FIG. 6, a direction tilted from the current imaging direction toward the RAO direction has been decided as an imaging direction at the movement destination. In this case, the irradiation range decision unit 27 decides an irradiation range 31A as an irradiation range at the movement destination. The irradiation range 31A at the movement destination corresponds to a direction tilted from the immediately preceding irradiation range 31F to the RAO direction. In addition, the irradiation range 31A at the movement destination is in the direction tilted to the RAO direction, entirely included in the low dose range, and located at a shortest distance 30va from the immediately preceding irradiation range 31F, thus satisfying the requirements (1), (2), and (3).

Note that if a skin dose corresponding to the partial range 31N is lower than the dose threshold, the irradiation range decision unit 27 may decide an irradiation range at the movement destination, with the partial range 31N being also included in the low dose range. In addition, a skin dose threshold (to be referred to as a low dose threshold hereinafter) to decide whether a given range is to be handled as a low dose range may be provided. A low dose threshold is set to a value lower than the above dose threshold. In this case, if a skin dose corresponding to the partial range 31N is lower than the low dose threshold, the irradiation range decision unit 27 handles the partial range 31N as a low dose range. On the other hand, if a skin dose corresponding to the partial range 31N is equal to or more than the low dose threshold, the irradiation range decision unit 27 does not handle the partial range 31N as a low dose range.

In addition, the irradiation range decision unit 27 may decide an irradiation range at the movement destination so as to satisfy a requirement (4) given below, in addition to the requirements (1) and (2). Note that the irradiation range decision unit 27 may decide an irradiation range at the movement destination so as to satisfy the requirements (1), (2), (3), and (4).

(4) An irradiation range at a movement destination does not overlap the immediately preceding irradiation range.

When deciding an irradiation range at a movement destination so as to satisfy the requirement (4), the irradiation range decision unit 27 decides an irradiation range at the movement destination so as not to overlap the immediately preceding irradiation range as shown in FIG. 3. Deciding an irradiation range at the movement destination so as not to overlap the immediately preceding irradiation range in this manner can disperse the skin doses of the subject. On the other hand, when deciding an irradiation range at the movement destination so as not to satisfy the requirement (4), the irradiation range decision unit 27 may decide an irradiation range at the movement destination so as to partially overlap the immediately preceding irradiation range as shown in FIG. 4. Since it is possible to decide an irradiation range at a movement destination, with skin doses being less than the dose threshold, so as to be close to the immediately preceding irradiation range in this manner, it is possible to suppress the angle change between fluoroscopic images and maintain the procedure efficiency.

The radiographic control circuitry 28 controls the respective units associated with an X-ray fluoroscopy operation. More specifically, the imaging control circuitry 28 controls the C-arm motor 13 to move the radiography system in an imaging direction corresponding to the irradiation range at the movement destination decided by the irradiation range decision unit 27. The radiographic control circuitry 28 outputs a movement control signal to the C-arm motor 13 in response to a timing when a skin dose in the current (immediately preceding) irradiation range becomes equal to or more than the dose threshold.

FIG. 8 is the first supplementary explanatory view for explaining processing by the radiographic control circuitry 28 according to the embodiment. FIG. 8 corresponds to FIG. 7. Assume that the irradiation range 31F of the subject P is currently irradiated with X-rays. In the positional relationship in the radiography system in this case, an X-ray generator 14F corresponds to the X-ray generator 14, and an X-ray detector 15F corresponds to the X-ray detector 15. Assume also that the current imaging direction is RAO/LAO 180°. In addition, assume that the irradiation range at the movement destination decided by the irradiation range decision unit 27 is the irradiation range 31B, and the imaging direction corresponding to the irradiation range 31B at the movement destination is LAO 170°. Assume that, referring to FIG. 8, an isocenter 331 of the radiography system has been aligned with a central position 32C of a region 32 of interest.

The radiographic control circuitry 28 controls the C-arm motor 13 to move the radiography system from a position corresponding to the current irradiation range to a position corresponding to the irradiation range at the movement destination in response to a timing when a skin dose corresponding to the current (immediately preceding) irradiation range reaches the dose threshold. In this case, the radiographic control circuitry 28 controls the C-arm motor 13 to move the C-arm 12 so as to avoid the central position 32C of the region 32 of interest from shifting on a fluoroscopic image before and after the movement.

In the case shown in FIG. 8, the radiographic control circuitry 28 controls the C-arm motor 13 to move the radiography system from the position (imaging direction RAO/LAO 180°) corresponding to the current irradiation range 31F to the position (imaging direction LAO 170°) corresponding to the irradiation range 31B at the movement destination. With this operation, the C-arm 12 is rotated through 10° in the LAO direction. As the C-arm 12 rotates, the X-ray generator 14 is moved from the X-ray generator 14F to an X-ray generator 14B, and the X-ray detector 15 is moved from the X-ray detector 15F to an X-ray detector 15B. Note that in the case shown in FIG. 8, since the isocenter 331 of the radiography system has been aligned with the central position 32C of the region 32 of interest, it is possible to change the imaging direction while maintaining the central position 32C of the region 32 of interest on the fluoroscopic image by only rotations about the rotation shafts of the C-arm support frame.

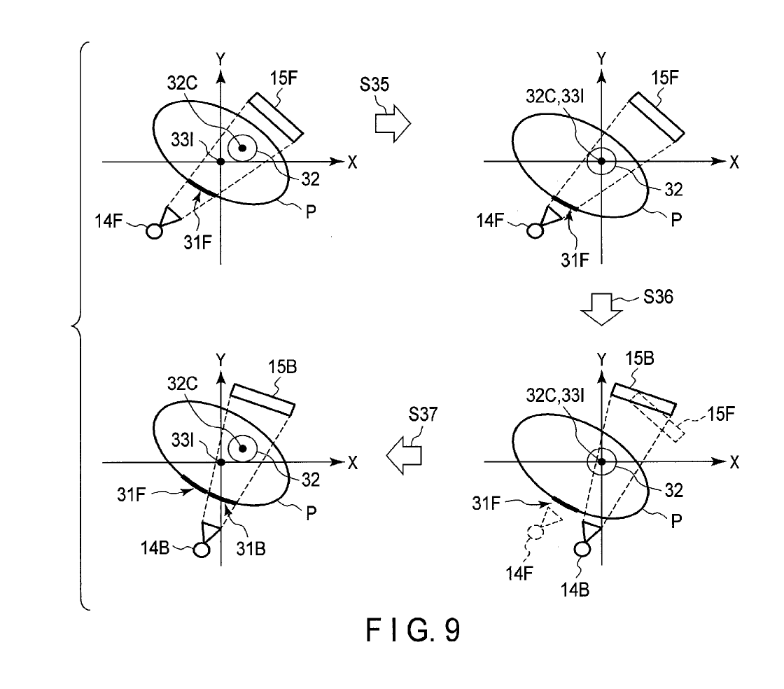

Control to be performed by the radiographic control circuitry 28 when the isocenter 331 of the radiography system has not been aligned with the central position 32C of the region 32 of interest will be described with reference to FIG. 9.

FIG. 9 is the second supplementary explanatory view for explaining processing by the radiographic control circuitry 28 according to this embodiment. Referring to FIG. 9, the isocenter 331 of the radiography system has not been aligned with the central position 32C of the region 32 of interest. Assume that the current irradiation range is the irradiation range 31F, and the irradiation range at the movement destination is the irradiation range 31B. The radiographic control circuitry 28 moves the radiography system to an imaging position corresponding to the irradiation range at the movement destination based on the relative positional relationship between the central position of the region of interest and the isocenter of the radiography system. Referring to FIG. 9, first of all, the radiographic control circuitry 28 specifies the direction and distance (to be referred to as the relative positional relationship hereinafter) between the isocenter 331 and the central position 32C of the region 32 of interest, and aligns the isocenter 331 with the central position 32C of the region 32 of interest by moving the C-arm 12 or the top plate (step S35). The radiographic control circuitry 28 then rotates the C-arm 12 (step S36). In this case, the radiographic control circuitry 28 decides a rotation amount in consideration of the restoration of the relative positional relationship. Lastly, the relationship between the isocenter 331 and the central position 32C of the region 32 of interest is restored (step S37). With the above processing, when the isocenter 331 of the radiography system has not been aligned with the central position 32C of the region 32 of interest, the radiography system is moved from an imaging position corresponding to the immediately preceding irradiation range 31F to an imaging position corresponding to the irradiation range 31B at the movement destination. At this time, the central position 32C of the region 32 of interest is maintained on the fluoroscopic image. Note that in practice, in the processing in steps S35, S36, and S37, the internal processing of the apparatus is collectively performed to directly move the radiography system from an imaging position corresponding to the immediately preceding irradiation range 31F to an imaging position corresponding to the irradiation range 31B at the movement destination.

In order to execute X-ray fluoroscopy, the radiographic control circuitry 28 controls the driving unit, the high voltage generator 16, and the X-ray detector 15 so as to synchronously perform movement of the C-arm 12, generation of X-rays, and detection of X-rays in accordance with the conditions set by the condition setting unit. For example, in response to pressing of the fluoroscopy switch, the radiographic control circuitry 28 controls the high voltage generator 16 in accordance with the set fluoroscopy conditions. At this time, the radiographic control circuitry 28 generates fluoroscopic image data corresponding to the radiography system by controlling the C-arm support frame, the X-ray detector 15, and the X-ray generator 14 in synchronism with control on the high voltage generator 16.

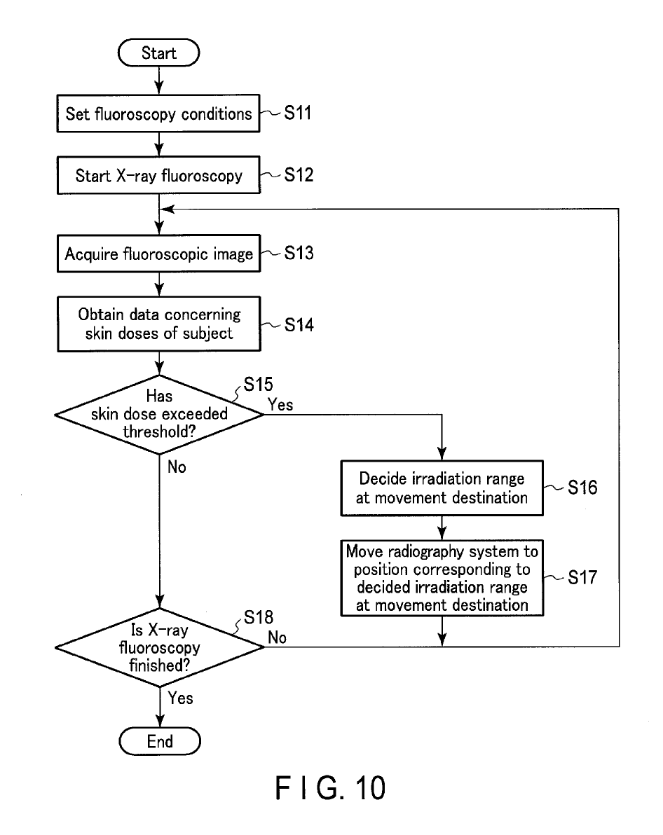

A workflow using the X-ray diagnostic apparatus 1 will be described below with reference to FIG. 10.

FIG. 10 is a flowchart for explaining a workflow using the X-ray diagnostic apparatus 1 according to this embodiment.

(Step S11) Fluoroscopy conditions are set.

The fluoroscopy conditions input by the user via the input interface circuitry 21 are set. In addition, the X-ray diagnostic apparatus 1 transmits the data of the set fluoroscopy conditions and the like to the exposure management system 70 via the communication circuitry 20.

(Step S12) X-ray fluoroscopy is started.

X-ray fluoroscopy is started in response to pressing of the fluoroscopy switch. In addition, the X-ray diagnostic apparatus 1 transmits a signal notifying the start of X-ray fluoroscopy to the exposure management system 70 via the communication circuitry 20.

(Step S13) A fluoroscopic images is acquired.

The X-ray diagnostic apparatus 1 acquires tomographic image data concerning the subject in accordance with set fluoroscopy conditions under the control of the radiographic control circuitry 28, and displays a fluoroscopic image on the display 26. The user performs a procedure such as insertion of a guide wire and a catheter into the patient while checking the fluoroscopic image displayed on the display 26.

(Step S14) Data concerning the skin doses of the subject are obtained.

In addition, data concerning the skin doses of the subject are obtained from the exposure management system 70 via the communication circuitry 20. The display 26 displays a screen indicating information concerning the skin doses of the subject.

(Step S15) It is determined whether a skin dose has exceeded a threshold.

The radiographic control circuitry 28 specifies a skin dose value corresponding to the current irradiation range. When the skin dose corresponding to the current irradiation range has reached the dose threshold, the process shifts to step S16. In contrast, if the skin dose corresponding to the current irradiation range is less than the dose threshold, the process shifts to step S18.

(Step S16) An irradiation range at a movement destination is decided.

The irradiation range decision unit 27 decides an irradiation range at a movement destination.

(Step S17) The radiography system is moved.

The radiographic control circuitry 28 moves the radiography system to a position corresponding to the irradiation range at the movement destination decided in step S16. The process then returns to step S13.

(Step S18) It is determined whether X-ray fluoroscopy is finished.

If the fluoroscopy switch is kept pressed, X-ray fluoroscopy is continued. In this case, the processing from step S13 to step S17 is repeatedly performed. If the fluoroscopy switch is released, X-ray fluoroscopy is finished.

The above processing from step S11 to step S18 is a workflow for X-ray fluoroscopy using the X-ray diagnostic apparatus 1. Note that step S16 (the procedure for deciding an irradiation range at a movement destination) may be performed before step S15 (the procedure for determining whether a skin dose has exceeded the threshold). This is because, since a skin dose does not increase outside the current irradiation range, the irradiation range at the movement destination to be decided does not change before and after the determined in step S15.

Using the X-ray diagnostic apparatus 1 according to this embodiment makes it possible to change the current irradiation range on a subject by moving the radiography system before a skin dose corresponding to the irradiation range exceeds the dose threshold. Although moving the radiography system will cause a change in fluoroscopic image, using the X-ray diagnostic apparatus 1 according to the embodiment can suppress the influence of the change in fluoroscopic image on the procedure performed by the user. This is because the X-ray diagnostic apparatus 1 according to the embodiment can decide an irradiation range at a movement destination close to the current (immediately preceding) irradiation range. In other words, since an imaging direction at the movement destination is decided to be close to the current (immediately preceding) imaging direction, the angle change between fluoroscopic images before and after the movement of the radiography system is small. Therefore, the user can observe fluoroscopic images before and after the movement of the radiography system without feeling any sense of discomfort. In this case, the fluoroscopic images obtained before and after the movement include a region of interest. It is preferable to maintain the central position of the region of interest on the fluoroscopic images obtained before and after the movement.

Using the X-ray diagnostic apparatus 1 can therefore disperse the skin doses of a subject while maintaining procedure efficiency comparable to that in the related art.

### Modification

This embodiment is configured to automatically move the radiography system to an imaging position corresponding to an irradiation range at a movement destination in response to a timing when a skin dose in the immediately preceding irradiation range has reached the dose threshold. The X-ray diagnostic apparatus 1 according to a modification of the embodiment is configured to change the irradiation range at the movement destination such that there is no partial range including any skin dose exceeding a dose threshold in the end in the interval between the start and the end of X-ray fluoroscopy. Therefore, the radiographic control circuitry 28 may move the radiography system at a timing before a skin dose in the immediately preceding irradiation range reaches the dose threshold.

Assume that the changing route of an irradiation range like that shown in FIG. 5 is decided. The irradiation range decision unit 27 decides an irradiation range at the movement destination so as to continuously or intermittently change the irradiation range in accordance with the changing route 50 of the irradiation range. According to the modification, therefore, the radiography system is continuously moved in accordance with the irradiation range which continuously changes. The imaging control circuitry 28 decides the moving velocity of the radiography system in this case based on the skin doses of the subject, fluoroscopy conditions, and a dose threshold. The radiographic control circuitry 28 can derive an increase in skin dose per unit time based on the fluoroscopy conditions. The radiographic control circuitry 28 can therefore continuously move the radiography system such that there is no range including any skin dose reaching the dose threshold even in a case in which the irradiation range continuously changes.

Note that the irradiation range decision unit 27 may decide an irradiation range at the movement destination so as to intermittently change the irradiation range in accordance with the changing route 50 of the irradiation range. In this case, the radiography system is intermittently moved in accordance with the irradiation range which intermittently changes. The radiographic control circuitry 28 decides the intervals at which the radiography system is moved, based on the skin doses of the subject, fluoroscopy conditions, and a dose threshold. The radiographic control circuitry 28 can move the radiography system at predetermined intervals such that there is no range including any skin dose reaching the dose threshold even in a case in which the irradiation range intermittently changes.

The X-ray diagnostic apparatus 1 according to this modification can reduce a change in the position of the radiography system before and after movement as compared with the X-ray diagnostic apparatus 1 according to this embodiment. Therefore, although the angle of a fluoroscopic image changes little by little, the user can perform a procedure while seeing the fluoroscopic image without concern for the angle change. In addition, the X-ray diagnostic apparatus 1 according to the modification can change the irradiation range at the movement destination such that there is no partial range including any skin dose exceeding a dose threshold in the end in the interval between the start and the end of X-ray fluoroscopy. It is therefore possible to disperse the skin doses of the subject.

Note that this embodiment and this modification have exemplified the single-plane X-ray diagnostic apparatus as a typical example of the present invention. However, the embodiment and the modification can also be applied to a biplane X-ray diagnostic apparatus including two radiography systems (the first and second radiography systems). In this case, the first irradiation range corresponding to the first radiography system and the second irradiation range corresponding to the second radiography system are generated on a subject. However, according to the embodiment and the modification, before skin doses corresponding to the current first and second irradiation ranges on the subject exceed a dose threshold, the radiography systems are independently moved to change the irradiation ranges. This can prevent the skin doses of the subject from locally concentrating. In addition, it is possible to make settings in advance to prevent the first and second irradiation ranges from overlapping each other. This can prevent skin doses from locally concentrating.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray diagnostic apparatus comprising:
a radiography system including an X-ray tube which generates X-rays and an X-ray detector which detects X-rays generated from the X-ray tube and transmitted through a subject and configured to radiograph the subject;
a support frame configured to movably support the radiography system;
communication circuitry configured to obtain skin dose information of the subject;
processing circuitry configured to decide an irradiation range at a movement destination of the radiography system based on the skin dose information of the subject; and
control circuitry configured to control the support frame to move the radiography system to a radiography position corresponding to the decided irradiation range at the movement destination,
wherein the irradiation range at the movement destination is close to an irradiation range on the subject immediately before the movement, and a skin dose corresponding to the irradiation range at the movement destination is less than a preset threshold.

2. The apparatus of claim 1, wherein the irradiation range at the movement destination is decided in a range at an angle less than 180° from the irradiation range immediately before the movement.

3. The apparatus of claim 1, wherein the irradiation range at the movement destination does not overlap the immediately preceding irradiation range.

4. The apparatus of claim 1, wherein the control circuitry controls the support frame in response to a timing when a skin dose in the immediately preceding irradiation range becomes a dose threshold.

5. The apparatus of claim 1, further comprising input circuitry configured to input a tilting direction of the support frame,
wherein the processing circuitry decides an irradiation range at the movement destination based on the immediately preceding irradiation range and the tilting direction.

6. The apparatus of claim 4, further comprising a display configured to display skin dose information of the subject on a display screen,
wherein the tilting direction is input in accordance with a user operation on the display screen.

7. The apparatus of claim 1, further comprising memory circuitry configured to store data concerning a changing route of the irradiation range,
wherein the processing circuitry decides an irradiation range at the movement destination based on the changing route, the immediately preceding irradiation range, and a history of the changing route.

8. The apparatus of claim 1, wherein the processing circuitry decides an irradiation range at the movement destination based on the immediately preceding irradiation range and a tiling direction, and
as the tilting direction, a direction tilted from an imaging direction corresponding to the immediately preceding irradiation range to a zenith direction of the top plate on which the subject is placed is decided.

9. The apparatus of claim 1, wherein the processing circuitry decides an irradiation range at the movement destination based on the immediately preceding irradiation range and the tilting direction, and
as the tilting direction, a direction tiled from an imaging direction corresponding to the immediately preceding irradiation range to an imaging direction corresponding to an initial irradiation range.

10. The apparatus of claim 1, wherein the apparatus comprises a biplane system including the two radiography systems, and
when irradiation fields of the two radiography systems overlap each other, the control circuitry fixes one radiography system and moves the other moving system.

11. An X-ray diagnostic apparatus comprising:
a radiography system including an X-ray tube which generates X-rays and an X-ray detector which detects X-rays generated from the X-ray tube and transmitted through a subject and configured to radiograph the subject;
a support frame configured to movably support the radiography system;
communication circuitry configured to acquire skin dose information of the subject;
processing circuitry configured to specify a range in which a skin dose of the subject is less than a preset threshold from the skin dose information of the subject and decide an irradiation range at a movement destination of the radiography system from a range in which the specified skin dose of the subject is less than the threshold; and
control circuitry configured to control the support frame to move the radiography system to an radiography position corresponding to the decided irradiation range at the movement destination.
